(19) United States
(12) Patent Application Publication
(10) Pub. No.: US 2021/0052232 A1
(43) Pub. Date: Feb. 25, 2021

(54) COMPUTED TOMOGRAPHY IMAGING MODULE WITH MULTIPLEXING

(71) Applicant: Analog Devices, Inc., Norwood, MA (US)

(72) Inventors: Hung D. NGO, Waltham, MA (US); Gary CARREAU, West Charleston, VT (US); Matthew A. WOODRUFF, Medford, MA (US)

(73) Assignee: Analog Devices, Inc., Norwood, MA (US)

(21) Appl. No.: 17/000,044

(22) Filed: Aug. 21, 2020

Related U.S. Application Data
(60) Provisional application No. 62/891,073, filed on Aug. 23, 2019.

(51) Int. Cl. A61B 6/03; A61B 6/00

(52) U.S. Cl. CPC A61B 6/032; A61B 6/4241

(57) ABSTRACT

There is disclosed herein a computed tomography (CT) imaging module. The CT imaging module may have a light sensor array to detect photons within a CT imaging system and may produce a plurality of signals based on the detected photons. A switching/multiplexing element may receive the plurality of signals and multiplex the plurality of signals for signal processing. The multiplexing of the plurality of signals may provide for less interconnections for the light sensors within the light sensor array, thereby reducing the risk of reliability failure of the interconnections. Further, fewer signal processing circuitry elements may be utilized for processing the plurality of signals, allowing the CT imaging module to take up less space.

### PRIORITY DATA

This application received benefit from and/or claim priority to U.S. Provisional Patent Application Ser. No. 62/891,073, filed Aug. 23, 2019 entitled, “COMPUTED TOMOGRAPHY IMAGING MODULE WITH MULTIPLEXING” incorporated herein by reference in its entirety.

### FIELD OF THE DISCLOSURE

This disclosure relates in general to the field of photon-to-digital conversion, and more particularly, though not exclusively, to a photon-to-digital converter module that may be utilized for computed tomography imaging.

### BACKGROUND

In computed tomography (CT) imaging, signal chains (including light sensors) are utilized to capture X-ray measurements to produce cross-sectional images of a scanned object. In legacy CT imaging systems, portions of the signal chains are located on multiple boards spread throughout a large system.

### BRIEF DESCRIPTION OF THE DRAWINGS

The present disclosure is best understood from the following detailed description when read with the accompanying figures. It is emphasized that, in accordance with the standard practice in the industry, various features are not necessarily drawn to scale, and are used for illustration purposes only. Where a scale is shown, explicitly or implicitly, it provides only one illustrative example. In other embodiments, the dimensions of the various features may be arbitrarily increased or reduced for clarity of discussion.

FIG. 1 illustrates an example computed tomography imaging module, according to various embodiments of the disclosure.

FIG. 2 illustrates another example computed tomography imaging module, according to various embodiments of the disclosure.

FIG. 3 illustrates another example computed tomography imaging module, according to various embodiments of the disclosure.

FIG. 4 illustrates a top view of an example CT imaging module, according to various embodiments of the disclosure.

### SUMMARY OF THE DISCLOSURE

There is disclosed herein a computed tomography (CT) imaging module. The CT imaging module may have a light sensor array to detect photons within a CT imaging system and may produce a plurality of signals based on the detected photons. A switching/multiplexing element may receive the plurality of signals and multiplex the plurality of signals for signal processing. The multiplexing of the plurality of signals may provide for less interconnections for the light sensors within the light sensor array, thereby reducing the risk of reliability failure of the interconnections. Further, fewer signal processing circuitry elements may be utilized for processing the plurality of signals, allowing the CT imaging module to take up less space.

A computed tomography (CT) imaging module is disclosed in embodiments herein. The CT imaging module may include a first substrate and a light sensor array coupled to the first substrate, the light sensor array to detect photons and output a plurality of signals that indicate characteristics of the photons. The CT imaging module may include a switching/multiplexing element coupled to the light sensor array, the switching/multiplexing element to multiplex the plurality of signals to produce a multiplexed signal. The CT imaging module may further include a second substrate coupled to the first substrate and signal processing circuitry coupled to the second substrate, the signal processing circuity located on an opposite side of the second substrate from the light sensor array and the switching/multiplexing element, the signal processing circuitry to perform signal processing of the multiplexed signal.

A CT imaging module subassembly is disclosed in embodiments herein. The CT imaging module may include a substrate and a light sensor array coupled to the substrate, the light sensor array to detect photons and output a plurality of signals that indicate characteristics of the photons. The CT imaging module subassembly may further include a switching/multiplexing element coupled to the light sensor array, the switching/multiplexing element to multiplex the plurality of signals for provision to signal processing circuitry for processing.

A CT imaging module is disclosed in embodiments herein. The CT imaging module may include a first substrate and a means for detecting photons coupled to the first substrate, the means for detecting photons to output a plurality of signals that indicate characteristics of photons detected by the means for detecting photons. The CT imaging module may include a means for multiplexing signals coupled to the means for detecting photons, the means for multiplexing signals to multiplex the plurality of signals to produce a multiplexed signal. The CT imaging module may further include a second substrate coupled to the first substrate and a means for processing signals coupled to the second substrate, the means for processing signals to process the multiplexed signal.

### DETAILED DESCRIPTION

The following disclosure provides many different embodiments, or examples, for implementing different features of the present disclosure. Specific examples of components and arrangements are described below to simplify the present disclosure. These are, of course, merely examples and are not intended to be limiting. Further, the present disclosure may repeat reference numerals and/or letters in the various examples, or in some cases across different figures. This repetition is for the purpose of simplicity and clarity and does not in itself dictate a specific relationship between the various embodiments and/or configurations discussed. Different embodiments may have different advantages, and no particular advantage is necessarily required of any embodiment.

The present disclosure illustrates computed tomography (CT) imaging modules that may be implemented in a CT imaging system in a tiled arrangement. Each of the CT imaging modules may include a CT signal chain, including a plurality of light sensors (such as photodiodes), a switching/multiplexing element, shielding material, and signal processing circuitry. Each CT imaging module can have a first substrate with a plurality of light sensors and the switching/multiplexing element can be located on opposite sides of the first substrate. The first substrate may have one or more vias located within the first substrate that provide for coupling between the plurality of light sensors on the side of the first substrate and the switching/multiplexing element located on the opposite side of the first substrate. A second substrate may be coupled to the first substrate, the second substrate having signal conversion circuitry located on an opposite side of the second substrate from the first substrate. The second substrate may include shielding material to shield the signal conversion circuitry.

In other embodiments, the CT imaging module may include the plurality of light sensors and the switching/multiplexing element implemented within an integrated circuit (IC). For example, the plurality of light sensors and the switching/multiplexing element may be located at a side of the first substrate. In some embodiments, one or more vias may be located within the first substrate and extend through the substrate to carry signals from the element with the plurality of light sensors and the switching/multiplexing element to the opposite side of the first substrate. From the opposite side of the first substrate, the signals may be routed to the signal conversion circuitry on the second substrate.

The switching/multiplexing element may multiplex signals received from the plurality of light sensors, where the signals produced by the plurality of light sensors may be based on photons detected by the plurality of light sensors. The multiplexing of the signals, as well as the ability to have a single set of signal processing circuitry to process the multiplexed signal rather than having signal processing circuitry for each signal, may provide benefits over legacy CT imaging systems. For example, the density of pixels of the plurality of light sensors may be greater. In some embodiments, a pixel pitch may be less than 1 millimeter for the plurality of light sensors described throughout this disclosure. Further, the CT imaging modules described throughout this disclosure may be reliable. In particular, the CT imaging modules may be more reliable than legacy CT imaging systems that attempt to provide the same pixel pitch and/or other characteristics of the CT imaging modules described herein.

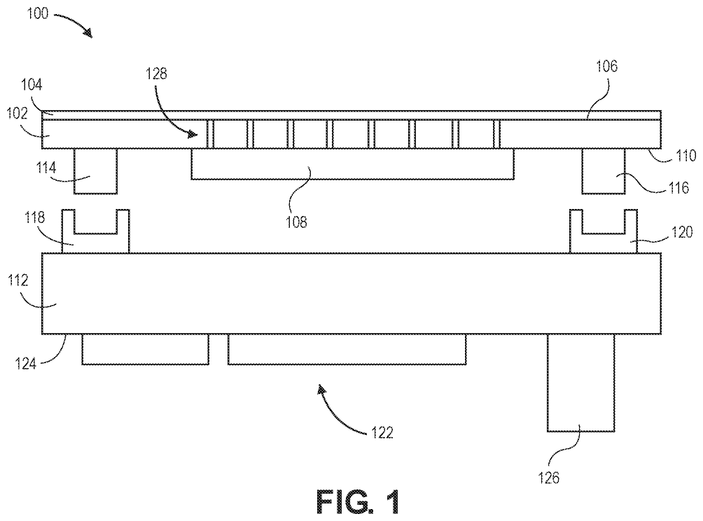

FIG. 1 illustrates an example CT imaging module 100, according to various embodiments of the disclosure. The CT imaging module 100 may perform photon-to-digital conversion, which may be utilized for CT imaging. Multiple CT imaging modules, having the features of the CT imaging module 100, may be implemented in a CT imaging system in a tiled arrangement to facilitate CT imaging. The CT imaging modules may be tiled together in x and y directions within the CT imaging system, where an entire CT imaging module fits within the x-y area of the corresponding light sensor array. As the resolution of CT imaging systems continue to increase, the ability to fit the entire CT imaging module within the x-y area of the corresponding light sensor array can allow the elements of a CT signal chain to fit in a smaller area than elements of legacy CT signal chains.

The CT imaging module 100 may include a substrate 102. The substrate 102 may comprise a die with one or more vias 128 that extend through the substrate 102. In some embodiments, the vias 128 may comprise through-silicon-vias (TSVs). The vias 128 may extend from a first side 106 of the substrate 102 to a second side 110 of the substrate 102. The vias 128 may be formed of electrically-conductive material and may be utilized for routing signals between the first side 106 of the substrate 102 and the second side 110 of the substrate 102. For example, the vias 128 may route signals between one or more elements located at the first side 106 of the substrate 102 and one or more elements located at the second side 110 of the substrate 102. While some vias 128 are illustrated in FIG. 1, and the vias 128 are illustrated as extending within a footprint of a switching/multiplexing element 108 located at the second side 110 of the substrate 102, it should be understood that there may be more or less vias 128 in other embodiments and the vias 128 may be located at different locations within the substrate 102 than shown in FIG. 1.

The CT imaging module 100 may further include a light sensor array 104 located at a first side 106 of the substrate 102. In some embodiments, the light sensor array 104 may extend for the entire first side 106 of the substrate 102. The light sensor array 104 may comprise a plurality of light sensors located on the first side 106 of the substrate 102 or the substrate 102 can comprise a bare die with the plurality of light sensors located at the first side 106 of the substrate 102 (i.e., the plurality of light sensors being incorporated into the substrate 102 at the first side 106 of the substrate 102). The light sensor array 104 may comprise a photodiode array in some embodiments, the photodiode array including one or more photodiodes. The light sensor array 104 may detect photons and output data indicating the intensities, wavelengths, and/or other characteristics of the photons detected.

The light sensor array 104 may be coupled to at least a portion of the vias 128 at the first side 106 of the substrate 102. The light sensor array 104 may produce one or more electrical signals (for example, the data) that represent the intensities, wavelengths, and/or other characteristics of the photons detected by the light sensor array 104. The light sensor array 104 may provide the signals to the vias 128 for transmission from the first side 106 of the substrate 102 to the second side 110 of the substrate 102.

The CT imaging module 100 may further include a switching/multiplexing element 108 located at a second side 110 of the substrate 102, the second side 110 being opposite to the first side 106. The switching/multiplexing element 108 may be located on the second side 110 of the substrate 102 or may be implemented in a die that is directly attached to the substrate 102 (such as a die-to-die bond) at the second side 110 of the substrate 102. The switching/multiplexing element 108 may comprise one or more switches, one or more multiplexers, or some combination thereof. The switching/multiplexing element 108 may be coupled to the light sensor array 104, where the switching/multiplexing element 108 can multiplex the outputs of each of the light sensors in the light sensor array 104. For example, the switching/multiplexing element 108 may be coupled to multiple light sensors in the light sensor array 104 (such as 32 light sensors, 40 light sensors, 64 light sensors, or 80 light sensors) by the vias 128, receive data from each of the multiple light sensors in the light sensor array 104, multiplex the received data to produce multiplexed data, and output the multiplexed data on a single output. It should be understood that the number of light sensors being multiplexed by the switching/multiplexing element 108 can be any number of light sensors greater than one in embodiments. Further, the switching/multiplexing element 108 may output multiplexed data on multiple outputs in other embodiments, where each of the multiple outputs may have a corresponding portion of the light sensors that provides the data for the output. The combination of the substrate 102, the light sensor array 104, and the switching/multiplexing element 108 may be referred to as a CT imaging module subassembly.

In other embodiments, the light sensor array 104 may be located at the second side 110 of the substrate 102. In these embodiments, the substrate 102 may comprise a material and/or may be of a thickness to allow photons to pass through the substrate 102, where the light sensor array 104 may detect the photons that have passed through the substrate 102 and produce signals based on the detected photons. The light sensor array 104 may be located between the substrate 102 and the switching/multiplexing element 108. Further, the vias 128 may be omitted in these embodiments.

The CT imaging module 100 may further include another substrate 112. The substrate 112 may be coupled to the substrate 102 via one or more connectors (such as bumps, connectors, and/or contacts) coupled to the substrate 102 and/or the substrate 112. For example, a first connector 114 and a second connector 116 coupled to the substrate 102 and a first connector 118 and a second connector 120 coupled to the substrate 112 may be utilized to couple the substrate 102 and the substrate 112 in the illustrated embodiment. The substrate 112 can be coupled at the second side 110 of the substrate 102. Accordingly, the substrate 112 may be located on an opposite side of the substrate 102 from the light sensor array 104, and the switching/multiplexing element 108 may be located between the substrate 102 and the substrate 112 in some embodiments. The substrate 112 may comprise shielding material, which may be in a shielding laminate form in some embodiments. In some embodiments, the shielding material may comprise X-ray shielding material.

The CT imaging module 100 may include signal processing circuitry 122 coupled to the substrate 112. The signal processing circuitry 122 may be coupled to a side 124 of the substrate 112 opposite to the substrate 102. Accordingly, the signal processing circuitry 122 is located on an opposite side of the substrate 112 from the light sensor array 104 and the switching/multiplexing element 108. The shielding material of the substrate 112 may shield the signal processing circuitry 122 from interference that may be caused by x-ray radiation (such as x-ray radiation that is incident on the light sensor array 104).

The signal processing circuitry 122 may be coupled to the switching/multiplexing element 108 and may receive the multiplexed data produced by the switching/multiplexing element 108. The signal processing circuitry 122 may be coupled to the switching/multiplexing element 108 via the connectors and electrical conductors (such as the vias 128, traces, vias, conductive layers, flexible circuits, and/or laminate technology conductors) of the substrate 102 and the substrate 112. The number of connectors and electrical conductors coupling the signal processing circuitry 122 to the switching/multiplexing element 108 may be less, due to the multiplexing, than CT imaging systems that do not multiplex the data captured by the light sensor array 104. As the resolution of CT imaging systems has increased, routing of the signal from the light sensors has increased and pushed the limits of assembly technologies, which has increased the risk of reliability failure at the interconnections. The CT imaging module 100 implementing the multiplexing of the data from the light sensor array 104 reduces the interconnections between the switching/multiplexing element 108 and the signal processing circuitry 122, and reduces the routing and interconnect density, thereby reducing the risk of reliability failure of the interconnections. Further, the CT imaging module 100 may take up less space than CT imaging modules that do not implement due to the CT imaging module 100 having less connectors and electrical conductors.

The signal processing circuitry 122 may receive the multiplexed data from the switching/multiplexing element 108 and may perform signal processing on the multiplexed data. The signal processing performed by the signal processing circuitry 122 may include converting the multiplexed data to a digital representation of the data. The signal processing circuitry 122 may include one or more signal converters and/or one or more system control circuitry integrated circuits (ICs) that facilitate the signal processing. The signal processing circuitry 122 processing the multiplexed data from multiple light sensors allows for less signal converters and/or system control circuitry ICs to be utilized for processing the same amount of data as compared to CT imaging systems that have each signal converter and/or system control circuitry IC coupled to a single light sensor. The reduced number of signal converters and/or system control circuitry ICs of the CT imaging module 100 may provide for reduced total cost and a smaller form-factor as compared to legacy CT imaging systems. Further, the multiplexing of the data from the multiple light sensors may enable greater pixel density (i.e., increase resolution) as a greater number of light sensors may be included in a space as compared to legacy CT imaging systems. Having the data being multiplexed may also allow for less routing required for a number of light sensors as compared to legacy CT imaging systems that do not implement multiplexing, which can allow for simplified routing and interconnection to the signal processing circuitry 122.

The CT imaging module 100 may further include one or more external connectors (such as bumps, connectors, and/or contacts). For example, the CT imaging module 100 includes an external connector 126. The external connectors may allow for coupling of the CT imaging module 100 to external hardware and/or devices.

The CT imaging module 100 may be implemented in a CT imaging system in a tiled arrangement, where the CT imaging module 100 is tiled with multiple other CT imaging modules having the features of the CT imaging module 100. The elements of the CT imaging module 100 may be located within the x-y area of the light sensor array 104, thereby allowing the light sensor arrays of the tiled CT imaging modules to abut each other. In particular, the substrate 102, the switching/multiplexing element 108, the connectors, the substrate 112, the signal processing circuitry 122, the external connector 126, or some combination thereof, may be located within the x-y area of the light sensor array 104.

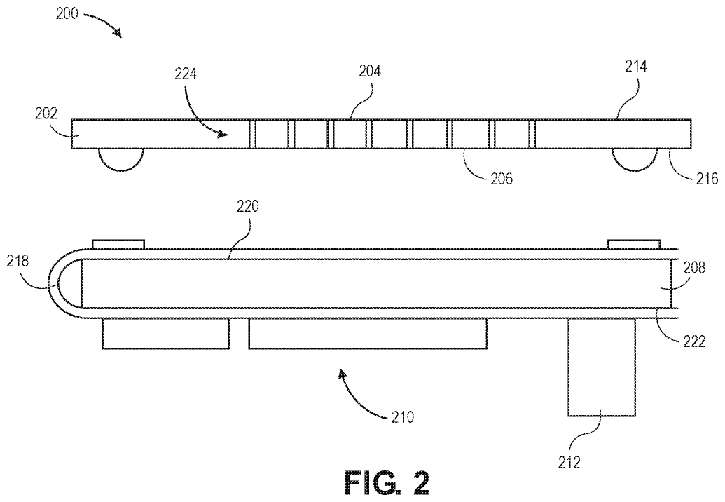

FIG. 2 illustrates another example CT imaging module 200, according to various embodiments of the disclosure. The CT imaging module 200 may include one or more features of the CT imaging module 100 (FIG. 1). For example, the CT imaging module 200 may include a substrate 202 having the features of the substrate 102 (FIG. 1), a light sensor array 204 having the features of the light sensor array 104 (FIG. 1), a switching/multiplexing element 206 having the features of the switching/multiplexing element 108 (FIG. 1), a substrate 208 having the features of the substrate 112 (FIG. 1), signal processing circuitry 210 having the features of the signal processing circuitry 122 (FIG. 1), and an external connector 212 having the features of the external connector 126 (FIG. 1) in embodiments. In some embodiments, the substrate 202 may comprise an integrated circuit (IC).

In the illustrated embodiment, the substrate 202 comprises a bare die, where the light sensor array 204 and the switching/multiplexing element 206 are coupled to the substrate 202 by being implemented in the substrate 202. In particular, the light sensor array 204 may be located at a first side 214 of the substrate 202 and may extend along a surface of the substrate 202. The switching/multiplexing element 206 may be located at a second side 216 of the substrate 202, the second side 216 opposite to the first side 214. The combination of the substrate 202, the light sensor array 204, and the switching/multiplexing element 206 may be referred to as a CT imaging module subassembly.

The substrate 202 may further include one or more vias 224. In some embodiments, the vias 224 may comprise TSVs. The vias 224 may extend from the first side 214 of the substrate 202 to the second side 216 of the substrate 202. The vias 224 may couple the light sensor array 204 located at the first side 214 of the substrate 202 to the switching/multiplexing element 206 located at the second side 216 of the substrate 202. The vias 224 may comprise electrically-conductive material and may provide for transmission of data (such as by electrical signals) between the light sensor array 204 and the switching/multiplexing element 206.

In other embodiments, the light sensor array 204 and the switching/multiplexing element 206 may both be located at the first side 214 of the substrate 202 or the second side 216 of the substrate 202. In the embodiments where the light sensor array 204 and the switching/multiplexing element 206 are both located at the second side 216 of the substrate 202, the substrate 202 may comprise a material and/or may be a thickness to allow photons to pass through the substrate 202. The light sensor array 204 may detect the photons that pass through the substrate and generate signals based on the detection of the photons. Further, the vias 224 may be omitted in embodiments where the light sensor array 204 and the switching/multiplexing element 206 are located at the second side 216 of the substrate 202. In embodiments where the light sensor array 204 and the switching/multiplexing element 206 are both located on the first side 214 of the substrate 202, the vias 224 may be utilized for transmission of the multiplexed data from the first side 214 of the substrate 202 to the second side 216 of the substrate 202.

Further, the illustrated embodiment of the CT imaging module 200 includes a flexible circuit 218. The flexible circuit 218 may be implemented as part of the substrate 208 or may be coupled to the substrate 208. The flexible circuit 218 may extend around a first side 220 of the substrate 208 and a second side 222 of the substrate 208, the second side 222 opposite to the first side 220, and route signals around the substrate 208. For example, the flexible circuit 218 may be utilized for routing multiplexed data from the switching/multiplexing element 206 to the signal processing circuitry 210 around the substrate 208. The substrate 208 may comprise shielding material, where the flexible circuit 218 is routed around the shielding material.

In other embodiments, the substrate 202 with the light sensor array 204 and the switching/multiplexing element 206 implemented within the substrate 202 may have other means for coupling the switching/multiplexing element 206 to the signal processing circuitry 210. For example, the substrate 202 may have vias, traces, conductive layers, and/or laminate technology conductors for transmission of multiplexed data from the switching/multiplexing element 206 to the signal processing circuitry 210. In these embodiments, the flexible circuit 218 may be omitted, or may be utilized along with the other means for transmission of the multiplexed data.

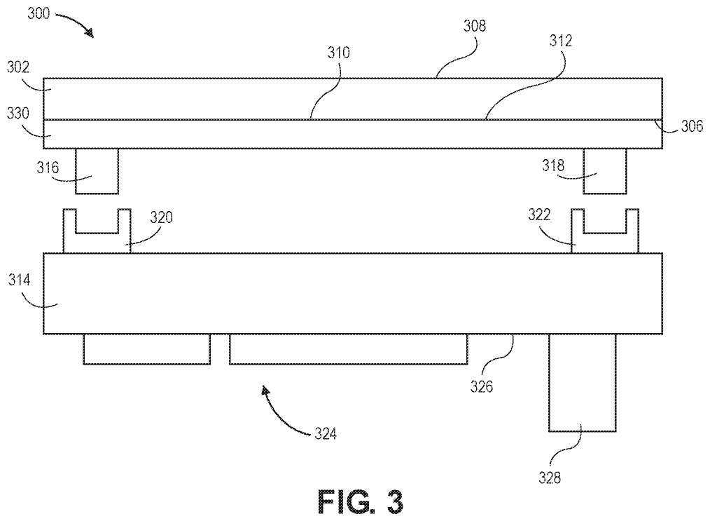

FIG. 3 illustrates another example CT imaging module 300, according to various embodiments of the disclosure. The CT imaging module 300 may perform photon-to-digital conversion, which may be utilized for CT imaging. Multiple CT imaging modules, having the features of the CT imaging module 300, may be implemented in a CT imaging system in a tiled arrangement to facilitate CT imaging. The CT imaging modules may be tiled together in x and y directions within the CT imaging system, where an entire CT imaging module fits within the x-y area of the corresponding light sensor array. As the resolution of CT imaging systems continue to increase, the ability to fit the entire CT imaging module within the x-y area of the corresponding light sensor array can allow the elements of a CT signal chain to fit in a smaller area than elements of legacy CT signal chains.

The CT imaging module 300 may include a substrate 302. The substrate 302 may include one or more of the features of the substrate 102 (FIG. 1) and/or the substrate 202 (FIG. 2). For example, the substrate 302 may comprise an IC or a bare die in some embodiments. The substrate 302 may comprise a material or may be of a thickness such that photons may pass through the substrate 302.

The CT imaging module 300 may further include a light sensor array 312. The light sensor array 312 may include one or more of the features of the light sensor array 104 (FIG. 1) and/or the light sensor array 204 (FIG. 2). The light sensor array 312 may be located at a first side 306 of the substrate 302 opposite to a second side 308 of the substrate 302 at which photons are to be directed. The photons may pass through the substrate 302 and may be detected by the light sensor array 312 located at the first side of the substrate 302. The light sensor array 312 may comprise a plurality of light sensors located on the first side 306 of the substrate 302 or the substrate 302 can comprise a bare die with the plurality of light sensors located at the first side 306 of the substrate 302 (i.e., the plurality of light sensors being incorporated into the substrate 302 at the first side 306 of the substrate 302). In the illustrated embodiment, the substrate 302 is illustrated as a bare die with the plurality of light sensors located at the first side 306 of the substrate 302. The light sensor array 312 may comprise a photodiode array in some embodiments, the photodiode array including one or more photodiodes. The light sensor array 312 may detect photons and output data indicating the intensities, wavelengths, and/or other characteristics of the photons detected.

The CT imaging module 300 may further include a switching/multiplexing element 310. The switching/multiplexing element 310 may be located at the first side 306 of the substrate 302. In some embodiments, the switching/multiplexing element 310 may be located between the light sensor array 312 and the first side 306 of the substrate 302, where both the switching/multiplexing element 310 and the light sensor array 312 are located at the first side 306 of the substrate 302. The switching/multiplexing element 310 may comprise one or more switches, one or more multiplexers, or some combination thereof. The switching/multiplexing element 310 may be coupled to the plurality of light sensors within the light sensor array 312 and may multiplex signals (i.e. transmission of the data) received from the plurality of light sensors. For example, the switching/multiplexing element 310 may be coupled to multiple light sensors in the light sensor array (such as 32 light sensors, 40, light sensors, 64 light sensors, or 80 light sensors) and may multiplex the signals from the multiple light sensors. The switching/multiplexing element 310 may output one or more signals produced from the multiplexed signals from the plurality of light sensors. In other embodiments, the CT imaging module 300 may include a plurality of switching/multiplexing elements, where each of the switching/multiplexing elements may be coupled to corresponding portions of the plurality of light sensors and may multiplex the signals from the corresponding portions of the plurality of light sensors. It should be understood that the number of light sensors being multiplexed by the switching/multiplexing element 310 can be any number of light sensors greater than one in embodiments. Further, the switching/multiplexing element 310 may output multiplexed data on multiple outputs in other embodiments, where each of the multiple outputs may have a corresponding portion of the light sensors that provides the data for the output. The combination of the substrate 302, the light sensor array 312, and the switching/multiplexing element 310 may be referred to as a CT imaging module subassembly.

The CT imaging module 300 may further include another substrate or interposer (referred to as interposer 330). The interposer 330 may be coupled to the first side 306 of the substrate 302. The interposer 330 may be affixed to the first side 306 of the substrate 302. In other embodiments, one or more connectors (such as bumps, connectors, and/or contacts) may couple the interposer 330 to the first side 306 of the substrate 302. The interposer 330 may comprise a rigid material and may provide rigidity to the substrate 302. For example, the substrate 302 may be thin in some embodiments to allow the photons to pass through the substrate 302, which may cause the substrate 302 to lack rigidity. Coupling the interposer 330 to the substrate 302 may provide rigidity for the substrate 302 that is lacking.

The CT imaging module 300 may further include another substrate 314. The substrate 314 may be coupled to the interposer 330 via one or more connectors (such as bumps, connectors, and/or contacts) coupled to the interposer 330 and/or the substrate 314. For example, a first connector 316 and a second connector 318 coupled to the interposer 330 and a first connector 320 and a second connector 322 coupled to the substrate 314 may be utilized to couple the interposer 330 and the substrate 314 in the illustrated embodiment. The substrate 314 can be coupled to an opposite side of the interposer 330 from the substrate 302. Accordingly, the substrate 314 may be located on an opposite side of the interposer 330 from the light sensor array 312 and the switching/multiplexing element 310 in some embodiments. The substrate 314 may comprise shielding material, which may be in a shielding laminate form in some embodiments. In some embodiments, the shielding material may comprise X-ray shielding material.

The CT imaging module 300 may include signal processing circuitry 324 coupled to the substrate 314. The signal processing circuitry 324 may be coupled to a side 326 of the substrate 314 opposite to the substrate 302 and the interposer 330. Accordingly, the signal processing circuitry 324 may be located on an opposite side of the substrate 314 from the light sensor array 312 and the switching/multiplexing element 310. The shielding material of the substrate 314 may shield the signal processing circuitry 324 from interference that may be caused by x-ray radiation (such as x-ray radiation that is incident on the light sensor array 312).

The signal processing circuitry 324 may be coupled to the switching/multiplexing element 310 and may receive the multiplexed data produced by the switching/multiplexing element 310. The signal processing circuitry 324 may be coupled to the switching/multiplexing element 310 via the connectors and electrical conductors (such as traces, vias, conductive layers, flexible circuits, and/or laminate technology conductors) of the substrate 302, the interposer 330, and the substrate 314. The number of connectors and electrical conductors coupling the signal processing circuitry 324 to the switching/multiplexing element 310 may be less, due to the multiplexing, than CT imaging systems that do not multiplex the data captured by the light sensor array 312. As the resolution of CT imaging systems has increased, routing of the signal from the light sensors has increased and pushed the limits of assembly technologies, which has increased the risk of reliability failure at the interconnections. The CT imaging module 300 implementing the multiplexing of the data from the light sensor array 312 reduces the interconnections between the switching/multiplexing element 310 and the signal processing circuitry 324, and reduces the routing and interconnect density, thereby reducing the risk of reliability failure of the interconnections. Further, the CT imaging module 300 may take up less space than CT imaging modules that do not implement due to the CT imaging module 300 having less connectors and electrical conductors.

The signal processing circuitry 324 may receive the multiplexed data from the switching/multiplexing element 310 and may perform signal processing on the multiplexed data. The signal processing performed by the signal processing circuitry 324 may include converting the multiplexed data to a digital representation of the data. The signal processing circuitry 324 may include one or more signal converters and/or one or more system control circuitry ICs that facilitate the signal processing. The signal processing circuitry 324 processing the multiplexed data from multiple light sensors allows for less signal converters and/or system control circuitry ICs to be utilized for processing the same amount of data as compared to CT imaging systems that have each signal converter and/or system control circuitry IC coupled to a single light sensor. The reduced number of signal converters and/or system control circuitry ICs of the CT imaging module 300 may provide for reduced total cost and a smaller form-factor as compared to legacy CT imaging systems. Further, the multiplexing of the data from the multiple light sensors may enable greater pixel density (i.e., increase resolution) as a greater number of light sensors may be included in a space as compared to legacy CT imaging systems. Having the data being multiplexed may also allow for less routing required for a number of light sensors as compared to legacy CT imaging systems that do not implement multiplexing, which can allow for simplified routing and interconnection to the signal processing circuitry 324.

The CT imaging module 300 may further include one or more external connectors (such as bumps, connectors, and/or contacts). For example, the CT imaging module 300 includes an external connector 328. The external connectors may allow for coupling of the CT imaging module 300 to external hardware and/or devices.

The CT imaging module 300 may be implemented in a CT imaging system in a tiled arrangement, where the CT imaging module 300 is tiled with multiple other CT imaging modules having the features of the CT imaging module 100, the CT imaging module 200, and/or the CT imaging module 300. The elements of the CT imaging module 300 may be located within the x-y area of the light sensor array 312, thereby allowing the light sensor arrays of the tiled CT imaging modules to abut each other. In particular, the substrate 302, the switching/multiplexing element 310, the connectors, the substrate 314, the interposer 330, the signal processing circuitry 324, the external connector 328, or some combination thereof, may be located within the x-y area of the light sensor array 312.

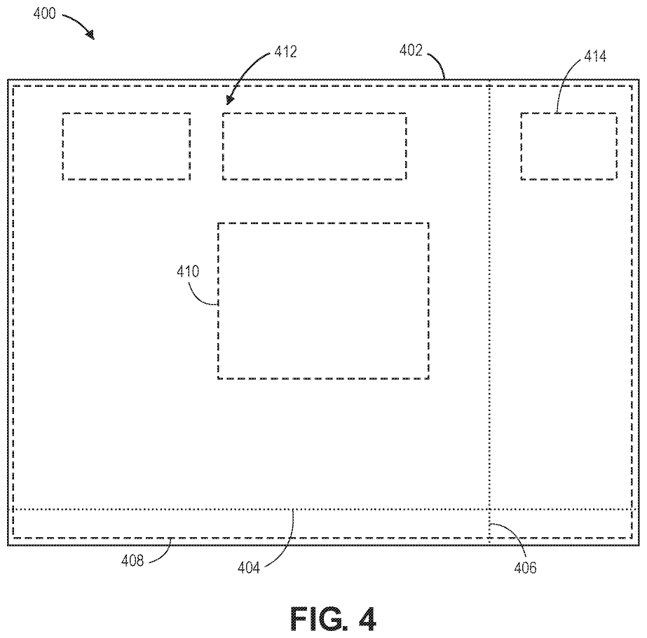

FIG. 4 illustrates a top view of an example CT imaging module 400, according to various embodiments of the disclosure. The CT imaging module 400 may include one or more of the features of the CT imaging module 100 (FIG. 1), the CT imaging module 200 (FIG. 2), and/or the CT imaging module 300 (FIG. 3).

The CT imaging module 400 may include a light sensor array 402. The light sensor array 402 may include one or more of the features of the light sensor array 104 (FIG. 1), the light sensor array 204 (FIG. 2), and the light sensor array 312 (FIG. 3). The light sensor array 402 is illustrated in solid lines to show that the light sensor array 402 is located at top surface of the CT imaging module 400. The light sensor array 402 may extend in an x-direction 404 and a y-direction 406. The outer edges of the light sensor array 402 may define an x-y area of the light sensor array 402.

The CT imaging module 400 may further include a first substrate and a second substrate, where the outlines of the first substrate and the second substrate are indicated by dashed line 408. The first substrate may include one or more of the features of the substrate 102 (FIG. 1), the substrate 202 (FIG. 2), and/or the substrate 302 (FIG. 3). The second substrate may include one or more of the features of the substrate 112 (FIG. 1), the substrate 208 (FIG. 2), and/or the substrate 314 (FIG. 3). The first substrate and the second substrate being shown in dashed lines indicates that first substrate and the second substrate is located behind the light sensor array 402 when viewed from the top of the CT imaging module 400. The first substrate and the second substrate may be located within the x-y area of the light sensor array 402, as shown by the dashed line 408 being with the x-y area. While the outer edges of the first substrate and the second substrate are located inside of the x-y area of the light sensor array 402 (as shown by the dashed line 408 being inside the x-area) for clarity, it should be understood that the outer edges of the first substrate and the second substrate may be aligned with the outer edges of the light sensor array 402 in other embodiments.

The CT imaging module 400 may further include a switching/multiplexing element 410. The switching/multiplexing element 410 may include one or more of the features of the switching/multiplexing element 108 (FIG. 1), the switching/multiplexing element 206 (FIG. 2), and/or the switching/multiplexing element 310 (FIG. 3). The switching/multiplexing element 410 being shown in dashed lines indicates that the switching/multiplexing element 410 is located behind the light sensor array 402 when viewed from the top of the CT imaging module 400. The switching/multiplexing element 410 may be located within the x-y area of the light sensor array 402, as shown by the outer edges of the switching/multiplexing element 410 being inside of the outer edges of the light sensor array 402.

The CT imaging module 400 may further include signal processing circuitry 412. The signal processing circuitry 412 may include one or more of the features of the signal processing circuitry 122 (FIG. 1), the signal processing circuitry 210 (FIG. 2), and/or the signal processing circuitry 324 (FIG. 3). The signal processing circuitry 412 being shown in dashed lines indicates that the signal processing circuitry 412 is located behind the light sensor array 402 when viewed from the top of the CT imaging module 400. The signal processing circuitry 412 may be located within the x-y area of the light sensor array 402, as shown by the outer edges of the signal processing circuitry 412 being inside of the outer edges of the light sensor array 402.

The CT imaging module 400 may further include an external connector 414. The external connector 414 may include one or more of the features of the external connector 126 (FIG. 1), the external connector 212 (FIG. 2), and/or the external connector 328 (FIG. 3). The external connector 414 being shown in dashed lines indicates that the external connector 414 is located behind the light sensor array 402 when viewed from the top of the CT imaging module 400. The external connector 414 may be located within the x-y area of the light sensor array 402, as shown by the outer edges of the external connector 414 being inside of the outer edges of the light sensor array 402.

While certain positions, sizes, and shapes of the light sensor array 402, the first substrate, the second substrate, the switching/multiplexing element 410, the signal processing circuitry 412, and the external connector 414 are illustrated in FIG. 4, it should be understood that the positions, sizes, and/or shapes of each of the elements may differ in other embodiments. In the other embodiments, the first substrate, the second substrate, the switching/multiplexing element 410, the signal processing circuitry 412, and/or the external connector 414 may be located within the x-y area of the light sensor array 402.

### EXAMPLE IMPLEMENTATIONS

The following examples are provided by way of illustration.

Example 1 may include a computed tomography (CT) imaging module, comprising a first substrate, a light sensor array coupled to the first substrate, the light sensor array to detect photons and output a plurality of signals that indicate characteristics of the photons, a switching/multiplexing element coupled to the light sensor array, the switching/multiplexing element to multiplex the plurality of signals to produce a multiplexed signal, a second substrate coupled to the first substrate, and signal processing circuitry coupled to the second substrate, the signal processing circuity located on an opposite side of the second substrate from the light sensor array and the switching/multiplexing element, the signal processing circuitry to perform signal processing of the multiplexed signal.

Example 2 may include the CT imaging module of example 1, wherein the switching/multiplexing element is located within the light sensor array.

Example 3 may include the CT imaging module of example 1, wherein the switching/multiplexing element and the signal processing circuitry are located within an x-y area of the light sensor array.

Example 4 may include the CT imaging module of example 1, wherein the second substrate comprises a shielding material.

Example 5 may include the CT imaging module of example 1, further comprising a flexible circuit that extends from a first side of the second substrate to a second side of the second substrate, wherein the flexible circuit is to route the multiplexed signal from the switching/multiplexing element to the signal processing circuitry.

Example 6 may include the CT imaging module of example 1, wherein the first substrate includes one or more through-silicon-vias (TSVs) to route one or more signals through the first substrate.

Example 7 may include the CT imaging module of example 6, wherein the light sensor array and the switching/multiplexing element are located at a first side of the first substrate, wherein the second substrate is located on a second side of the first substrate, the second side of the first substrate being opposite to the first side of the first substrate, and wherein the one or more TSVs are to be utilized to route the multiplexed signal from the switching/multiplexing element to the signal processing circuitry.

Example 8 may include the CT imaging module of example 6, wherein the light sensor array is located at a first side of the first substrate, wherein the switching/multiplexing element is located at a second side of the first substrate, the second side of the first substrate being opposite to the first side of the first substrate, and wherein the one or more TSVs are to be utilized to route the plurality of signals from the light sensor array to the switching/multiplexing element.

Example 9 may include the CT imaging module of example 1, further comprising an external connector coupled to the second substrate, the external connector to be utilized to couple the CT imaging module to a CT imaging system.

Example 10 may include the CT imaging module of example 1, wherein the first substrate comprises a bare die, and wherein the light sensor array is incorporated into the first substrate.

Example 11 may include a CT imaging module subassembly, comprising a substrate, a light sensor array coupled to the substrate, the light sensor array to detect photons and output a plurality of signals that indicate characteristics of the photons, and a switching/multiplexing element coupled to the light sensor array, the switching/multiplexing element to multiplex the plurality of signals for provision to signal processing circuitry for processing.

Example 12 may include the CT imaging module subassembly of example 11, wherein the substrate and the switching/multiplexing element are located within an x-y area of the light sensor array.

Example 13 may include the CT imaging module subassembly of example 11, wherein the switching/multiplexing element is located within the light sensor array.

Example 14 may include the CT imaging module subassembly of example 11, wherein the light sensor array and the switching/multiplexing element are located at a first side of the substrate, and wherein the substrate includes one or more through-silicon-vias (TSVs) that are to route signals from the switching/multiplexing element to a second side of the substrate.

Example 15 may include the CT imaging module subassembly of example 11, wherein the light sensor array is located at a first side of the substrate, wherein the switching/multiplexing element is located at a second side of the substrate, the second side of the substrate opposite to the first side of the substrate, and wherein the substrate includes one or more through-silicon-vias (TSVs) that are to route the plurality of signals from the light sensor array to the switching/multiplexing element.

Example 16 may include the CT imaging module subassembly of example 11, wherein the switching/multiplexing element is multiplex the plurality of signals from 32 light sensors, 40 light sensors, 64 light sensors, or 80 light sensors of the light sensor array.

Example 17 may include a computed tomography (CT) imaging module, comprising a first substrate, a means for detecting photons coupled to the first substrate, the means for detecting photons to output a plurality of signals that indicate characteristics of photons detected by the means for detecting photons, a means for multiplexing signals coupled to the means for detecting photons, the means for multiplexing signals to multiplex the plurality of signals to produce a multiplexed signal, a second substrate coupled to the first substrate, and a means for processing signals coupled to the second substrate, the means for processing signals to process the multiplexed signal.

Example 18 may include the CT imaging module of example 17, wherein the means for multiplexing signals is located within the means for detecting photons.

Example 19 may include the CT imaging module of example 17, wherein the means for multiplexing signals is located within an x-y area of the means for detecting photons.

Example 20 may include the CT imaging module of example 17, wherein the means for processing signals is located on an opposite side of the second substrate from the means for multiplexing signals, and wherein the second substrate comprises a shielding material.

The foregoing outlines features of one or more embodiments of the subject matter disclosed herein. These embodiments are provided to enable a person having ordinary skill in the art (PHOSITA) to better understand various aspects of the present disclosure. Certain well-understood terms, as well as underlying technologies and/or standards may be referenced without being described in detail. It is anticipated that the PHOSITA will possess or have access to background knowledge or information in those technologies and standards sufficient to practice the teachings of the present disclosure.

The PHOSITA will appreciate that they may readily use the present disclosure as a basis for designing or modifying other processes, structures, or variations for carrying out the same purposes and/or achieving the same advantages of the embodiments introduced herein. The PHOSITA will also recognize that such equivalent constructions do not depart from the spirit and scope of the present disclosure, and that they may make various changes, substitutions, and alterations herein without departing from the spirit and scope of the present disclosure.

Note that the activities discussed above with reference to the FIGURES are applicable to any integrated circuit that involves signal processing, particularly those that can execute specialized software programs or algorithms, some of which may be associated with processing digitized real-time data. Certain embodiments can relate to multi-DSP, multi-ASIC, or multi-SoC signal processing, floating point processing, signal/control processing, fixed-function processing, microcontroller applications, etc. In certain contexts, the features discussed herein can be applicable to medical systems, scientific instrumentation, wireless and wired communications, radar, industrial process control, audio and video equipment, current sensing, instrumentation (which can be highly precise), and other digital-processing-based systems. Moreover, certain embodiments discussed above can be provisioned in digital signal processing technologies for medical imaging, patient monitoring, medical instrumentation, and home healthcare. This could include, for example, pulmonary monitors, accelerometers, heart rate monitors, or pacemakers, along with peripherals therefor.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

The particular embodiments of the present disclosure may readily include a system on chip (SoC) central processing unit (CPU) package. An SoC represents an integrated circuit (IC) that integrates components of a computer or other electronic system into a single chip. It may contain digital, analog, mixed-signal, and radio frequency functions: all of which may be provided on a single chip substrate. Other embodiments may include a multi-chip-module (MCM), with a plurality of chips located within a single electronic package and configured to interact closely with each other through the electronic package. Any module, function, or block element of an ASIC or SoC can be provided, where appropriate, in a reusable “black box” intellectual property (IP) block, which can be distributed separately without disclosing the logical details of the IP block. In various other embodiments, the digital signal processing functionalities may be implemented in one or more silicon cores in application-specific integrated circuits (ASICs), field-programmable gate arrays (FPGAs), and other semiconductor chips.

In example implementations, at least some portions of the processing activities outlined herein may also be implemented in software. In some embodiments, one or more of these features may be implemented in hardware provided external to the elements of the disclosed figures, or consolidated in any appropriate manner to achieve the intended functionality. The various components may include software (or reciprocating software) that can coordinate in order to achieve the operations as outlined herein. In still other embodiments, these elements may include any suitable algorithms, hardware, software, components, modules, interfaces, or objects that facilitate the operations thereof.

Additionally, some of the components associated with described microprocessors may be removed, or otherwise consolidated. In a general sense, the arrangements depicted in the figures may be more logical in their representations, whereas a physical architecture may include various permutations, combinations, and/or hybrids of these elements. It is imperative to note that countless possible design configurations can be used to achieve the operational objectives outlined herein. Accordingly, the associated infrastructure has a myriad of substitute arrangements, design choices, device possibilities, hardware configurations, software implementations, equipment options, etc.

Any suitably-configured processor component can execute any type of instructions associated with the data to achieve the operations detailed herein. Any processor disclosed herein could transform an element or an article (for example, data) from one state or thing to another state or thing. In another example, some activities outlined herein may be implemented with fixed logic or programmable logic (for example, software and/or computer instructions executed by a processor) and the elements identified herein could be some type of a programmable processor, programmable digital logic (for example, an FPGA, an erasable programmable read only memory (EPROM), an electrically erasable programmable read only memory (EEPROM)), an ASIC that includes digital logic, software, code, electronic instructions, flash memory, optical disks, CD-ROMs, DVD ROMs, magnetic or optical cards, other types of machine-readable mediums suitable for storing electronic instructions, or any suitable combination thereof. In operation, processors may store information in any suitable type of non-transitory storage medium (for example, random access memory (RAM), read only memory (ROM), FPGA, EPROM, electrically erasable programmable ROM (EEPROM), etc.), software, hardware, or in any other suitable component, device, element, or object where appropriate and based on particular needs. Further, the information being tracked, sent, received, or stored in a processor could be provided in any database, register, table, cache, queue, control list, or storage structure, based on particular needs and implementations, all of which could be referenced in any suitable timeframe. Any of the memory items discussed herein should be construed as being encompassed within the broad term ‘memory.’ Similarly, any of the potential processing elements, modules, and machines described herein should be construed as being encompassed within the broad term ‘microprocessor’ or ‘processor.’ Furthermore, in various embodiments, the processors, memories, network cards, buses, storage devices, related peripherals, and other hardware elements described herein may be realized by a processor, memory, and other related devices configured by software or firmware to emulate or virtualize the functions of those hardware elements.

Computer program logic implementing all or part of the functionality described herein is embodied in various forms, including, but in no way limited to, a source code form, a computer executable form, a hardware description form, and various intermediate forms (for example, mask works, or forms generated by an assembler, compiler, linker, or locator). In an example, source code includes a series of computer program instructions implemented in various programming languages, such as an object code, an assembly language, or a high-level language such as OpenCL, RTL, Verilog, VHDL, Fortran, C, C++, JAVA, or HTML for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

In one example embodiment, any number of electrical circuits of the FIGURES may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), memory elements, etc. can be suitably coupled to the board based on particular configuration needs, processing demands, computer designs, etc. Other components such as external storage, additional sensors, controllers for audio/video display, and peripheral devices may be attached to the board as plug-in cards, via cables, or integrated into the board itself. In another example embodiment, the electrical circuits of the FIGURES may be implemented as standalone modules (e.g., a device with associated components and circuitry configured to perform a specific application or function) or implemented as plug-in modules into application-specific hardware of electronic devices.

Note that with the numerous examples provided herein, interaction may be described in terms of two, three, four, or more electrical components. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this disclosure. In certain cases, it may be easier to describe one or more of the functionalities of a given set of flows by only referencing a limited number of electrical elements. It should be appreciated that the electrical circuits of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

What is claimed is:

1. A computed tomography (CT) imaging module, comprising:
a first substrate;
a light sensor array coupled to the first substrate, the light sensor array to detect photons and output a plurality of signals that indicate characteristics of the photons;
a switching/multiplexing element coupled to the light sensor array, the switching/multiplexing element to multiplex the plurality of signals to produce a multiplexed signal;
a second substrate coupled to the first substrate; and
signal processing circuitry coupled to the second substrate, the signal processing circuity located on an opposite side of the second substrate from the light sensor array and the switching/multiplexing element, the signal processing circuitry to perform signal processing of the multiplexed signal.

2. The CT imaging module of claim 1, wherein the switching/multiplexing element is located within the light sensor array.

3. The CT imaging module of claim 1, wherein the switching/multiplexing element and the signal processing circuitry are located within an x-y area of the light sensor array.

4. The CT imaging module of claim 1, wherein the second substrate comprises a shielding material.

5. The CT imaging module of claim 1, further comprising a flexible circuit that extends from a first side of the second substrate to a second side of the second substrate, wherein the flexible circuit is to route the multiplexed signal from the switching/multiplexing element to the signal processing circuitry.

6. The CT imaging module of claim 1, wherein the first substrate includes one or more vias to route one or more signals through the first substrate.

7. The CT imaging module of claim 6, wherein the light sensor array and the switching/multiplexing element are located at a first side of the first substrate, wherein the second substrate is located on a second side of the first substrate, the second side of the first substrate being opposite to the first side of the first substrate, and wherein the one or more vias are to be utilized to route the multiplexed signal from the switching/multiplexing element to the signal processing circuitry.

8. The CT imaging module of claim 6, wherein the light sensor array is located at a first side of the first substrate, wherein the switching/multiplexing element is located at a second side of the first substrate, the second side of the first substrate being opposite to the first side of the first substrate, and wherein the one or more vias are to be utilized to route the plurality of signals from the light sensor array to the switching/multiplexing element.

9. The CT imaging module of claim 1, further comprising an external connector coupled to the second substrate, the external connector to be utilized to couple the CT imaging module to a CT imaging system.

10. The CT imaging module of claim 1, wherein the first substrate comprises a bare die, and wherein the light sensor array is incorporated into the first substrate.

11. A computed tomography (CT) imaging module subassembly, comprising:
a substrate;
a light sensor array coupled to the substrate, the light sensor array to detect photons and output a plurality of signals that indicate characteristics of the photons; and
a switching/multiplexing element coupled to the light sensor array, the switching/multiplexing element to multiplex the plurality of signals for provision to signal processing circuitry for processing.

12. The CT imaging module subassembly of claim 11, wherein the substrate and the switching/multiplexing element are located within an x-y area of the light sensor array.

13. The CT imaging module subassembly of claim 11, wherein the switching/multiplexing element is located within the light sensor array.

14. The CT imaging module subassembly of claim 11, wherein the light sensor array and the switching/multiplexing element are located at a first side of the substrate, and wherein the substrate includes one or more vias that are to route signals from the switching/multiplexing element to a second side of the substrate.

15. The CT imaging module subassembly of claim 11, wherein the light sensor array is located at a first side of the substrate, wherein the switching/multiplexing element is located at a second side of the substrate, the second side of the substrate opposite to the first side of the substrate, and wherein the substrate includes one or more vias that are to route the plurality of signals from the light sensor array to the switching/multiplexing element.

16. The CT imaging module subassembly of claim 11, wherein the switching/multiplexing element is multiplex the plurality of signals from 32 light sensors, 40 light sensors, 64 light sensors, or 80 light sensors of the light sensor array.

17. A computed tomography (CT) imaging module, comprising:
a first substrate;
a means for detecting photons coupled to the first substrate, the means for detecting photons to output a plurality of signals that indicate characteristics of photons detected by the means for detecting photons;
a means for multiplexing signals coupled to the means for detecting photons, the means for multiplexing signals to multiplex the plurality of signals to produce a multiplexed signal;
a second substrate coupled to the first substrate; and
a means for processing signals coupled to the second substrate, the means for processing signals to process the multiplexed signal.

18. The CT imaging module of claim 17, wherein the means for multiplexing signals is located within the means for detecting photons.

19. The CT imaging module of claim 17, wherein the means for multiplexing signals is located within an x-y area of the means for detecting photons.

20. The CT imaging module of claim 17, wherein the means for processing signals is located on an opposite side of the second substrate from the means for multiplexing signals, and wherein the second substrate comprises a shielding material.
